(19) United States
(12) Patent Application Publication
(10) Pub. No.: US 2022/0347331 A1
(43) Pub. Date: Nov. 3, 2022

(54) ROBOT FOR AUTOMATICALLY PREVENTING EPIDEMICS

(71) Applicant: HILLS ENGINEERING CO. LTD, (US)

(72) Inventors: Mueng Gyu PARK, Seongnam-si (KR); Jong Cheol YOON, Seongnam-si (KR)

(73) Assignee: HILLS ENGINEERING CO. LTD, Seongnam-si, Gyeonggi-do (KR)

(21) Appl. No.: 17/599,138

(22) PCT Filed: Nov. 16, 2020
(86) PCT No.: PCT/KR2020/016112

§ 371 Date: Sep. 28, 2021

(51) Int. Cl. A61L 2/22; A61L 2/10; A61L 2/24

(52) U.S. Cl. CPC A61L 2/22; A61L 2/10; A61L 2/24; A61L 2202/11; A61L 2202/14; A61L 2202/16; A61L 2202/25; A61L 2202/15

(57) ABSTRACT

A robot for automatically preventing epidemics includes: a moving body which is movable along a passage of an interior space of a transportation element; an injection unit which is mounted to the moving body and injects a chemical solution for disinfection toward the seat disposed in the interior space; and an ultraviolet ray irradiation unit which is mounted to a lower end of the moving body and irradiates ultraviolet rays toward a floor of the passage, in which the robot for automatically preventing epidemics automatically performs epidemic prevention work in the interior space.

### TECHNICAL FIELD

The present invention relates to a robot for automatically preventing epidemics, and more particularly, to a robot for automatically preventing epidemics which is capable of automatically performing epidemic prevention work on seats and the like while moving along a passage in an interior space of a transportation means, such as an airplane, thereby improving efficiency and accuracy of the epidemic prevention work.

### BACKGROUND ART

As the COVID-19 situation is a prolonged, concern about the infection of passengers trying to use transportation means, such as airplanes, trains, or buses, are increasing. Accordingly, airlines and KORAIL have further strengthened epidemic prevention and mobilize various methods to, for example, block in advance or minimize the infection within the airplane.

For example, an attempt is made to reduce the risk of in-flight infection by installing air curtains between seats in an airplane to allow the air in the airplane to move from the top to the bottom and then discharging the air to the outside.

In the meantime, airlines, such as Korea Airline, try to gain trust from customers by ensuring safety against infection by using an integrated epidemic prevention program, such as “Care First”. This is to provide a safe environment for customers through regular in-flight disinfection, activation of non-face-to-face procedures, temperature checks of passengers, maintaining distance between passengers, and strengthening in-flight hygiene, and the like.

However, in the case of the existing in-flight disinfection, since each in-flight part is sterilized by mobilizing people, efficiency of preventing epidemic is reduced, there is the convenience of having to mobilize manpower every time, and there is a limit in that quality of in-flight disinfection cannot be kept constant because the in-flight infection is performed by people.

Accordingly, there is a need for developing an automated robot for preventing epidemics having a new configuration that is capable of efficiency and accurately performing in-flight epidemic prevention.

As a related prior art, for example, Korean Patent No. 10-1724447 discloses a UV sterilization robot.

### DISCLOSURE

### Technical Problem

An exemplary embodiment of the present invention provides a robot for automatically preventing epidemic which is capable of automatically performing epidemic prevention work on seats and the like while moving along a passage in an interior space of a transportation means, such as an airplane, thereby improving efficiency and accuracy of the epidemic prevention work.

An object to be solved in the present invention is not limited to the aforementioned objects, and other objects not-mentioned herein will be clearly understood by those skilled in the art from descriptions below.

### Technical Solution

An exemplary embodiment of the present invention provides a robot for automatically preventing epidemics, the robot including: a moving body which is movable along a passage of an interior space having a seat; an injection unit which is mounted to the moving body and injects a chemical solution for disinfection toward the seat disposed in the interior space; and an ultraviolet ray irradiation unit which is mounted to a lower end of the moving body and irradiates ultraviolet rays toward a floor of the passage, in which the robot for automatically preventing epidemics automatically performs epidemic prevention work in the interior space.

Further, the injection unit may include a first arm mounted to the moving body so as to be slidable in a horizontal direction above the seat, and a second arm slidably movable with respect to the first arm, and the second arm may be provided with an injection nozzle which injects the chemical solution toward the seat in a radial direction.

Further, the injection unit may be tiltable in an upper direction and a lower direction with respect to a connection part with the moving body as an axis,

Further, the moving body may be provided with a guide member which guides the movement of the injection unit so that the injection unit is movable in an upper direction or a lower direction with respect to the moving body.

Further, the injection unit may include: first arms mounted to both sides of the moving body; and a second arm tiltably coupled to an upper end of the first arm, and the first arm and the second arm may be provided with a plurality of injection nozzles in a longitudinal direction.

Further, the injection unit may further include a third arm coupled to the second arm so as to be slidable with respect to the second arm, and the third arm may be provided with a plurality of injection nozzles in a longitudinal direction.

Further, the moving body may be mounted with a plurality of moving wheels at a lower end.

Further, the injection units may be provided in two and symmetrically mounted to both lateral walls of the moving body, and each of the injection units is individually operable.

Further, the robot for automatically preventing epidemics according to the exemplary embodiment of the present invention may further include: a detection unit which is mounted to the injection unit and detects a distance from an object disposed in the passage; and a control unit which controls an operation of the moving body or the injection unit based on detection information obtained by the detection unit.

Further, the detection unit may be mounted to the injection unit, and the control unit may control a tilting operation of the injection unit with respect to the moving body, a sliding operation of another portion with respect to one portion of the injection unit, and a sliding operation in a height direction of the injection unit with respect to the moving body based on information detected by the detection unit.

Further, the robot for automatically preventing epidemics according to the exemplary embodiment of the present invention may further include an additional injection unit which is mounted to the moving body and injects a chemical solution to a space between the seats provided in the passage.

Further, the robot for automatically preventing epidemics according to the exemplary embodiment of the present invention may automatically perform epidemic prevention work in a cabin space of an airplane, a transportation means with seats, or a performance hall with seats.

### Advantageous Effects

According to the exemplary embodiments of the present invention, the robot for automatically preventing epidemics is capable of automatically performing epidemic prevention work on a seat and the like while moving along a passage of an interior space of a transportation means, such as an airplane, thereby improving efficiency and accuracy of the epidemic prevention work.

Further, the robot for automatically preventing epidemics is capable of performing disinfection work by irradiating ultraviolet rays to a floor portion of the passage and the like.

### DESCRIPTION OF DRAWINGS

FIG. 1 is a diagram schematically illustrating the case where a robot for automatically preventing epidemics according to an exemplary embodiment of the present invention is used in a cabin of an airplane.

FIG. 2 is a diagram illustrating a schematic configuration of the robot for automatically preventing epidemics illustrated in FIG. 1.

FIG. 3 is a diagram illustrating a tilting operation of an injection unit of the robot for automatically preventing epidemics illustrated in FIG. 2.

FIG. 4 is a diagram illustrating a sliding movement operation of the injection unit of the robot for automatically preventing epidemics illustrated in FIG. 2.

FIG. 5 is a diagram illustrating a side surface of the robot for automatically preventing epidemics illustrated in FIG. 2, and schematically illustrates a principle of a movement between seats.

FIG. 6 is a perspective view schematically illustrating a configuration of a robot for automatically preventing epidemics according to another exemplary embodiment of the present invention.

FIG. 7 is a perspective view illustrating an operation of an injection unit illustrated in FIG. 6.

### BEST MODE

Advantages and characteristics, and a method for achieving them will be clear when exemplary embodiments described in detail with reference to the accompanying drawings are referred to. However, the present disclosure is not limited to exemplary embodiments disclosed herein but will be implemented in various forms, and the exemplary embodiments are provided so that the present disclosure is completely disclosed, and a person of ordinary skilled in the art can fully understand the scope of the present disclosure, and the present disclosure will be defined only by the scope of the appended claims. Throughout the specification, the same reference numeral indicates the same constituent element.

Hereinafter, the exemplary embodiments of the present invention will be described in detail with reference to the accompanying drawings.

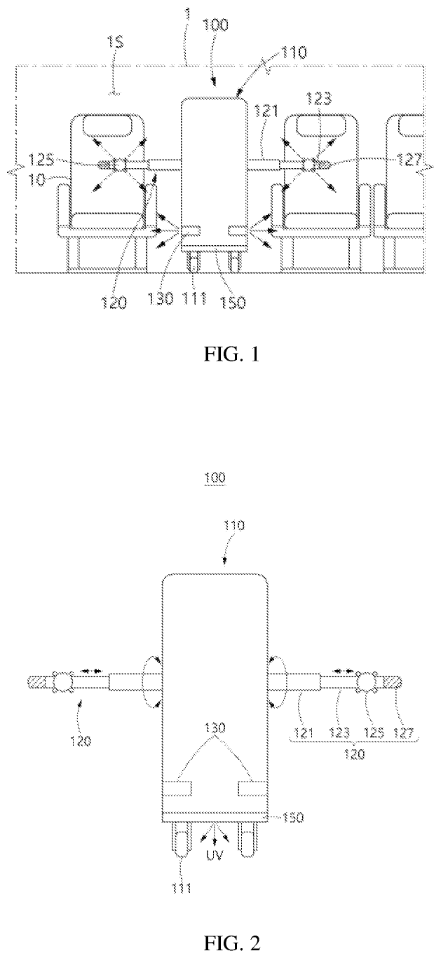

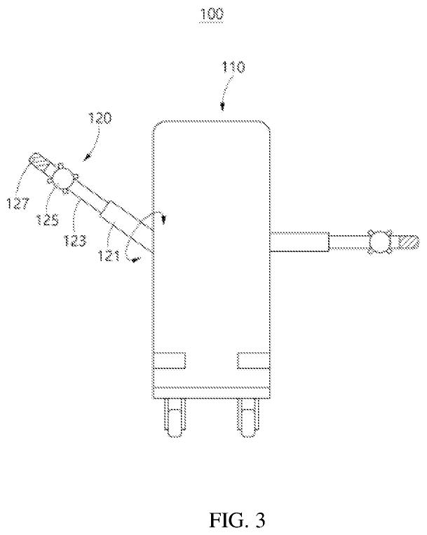

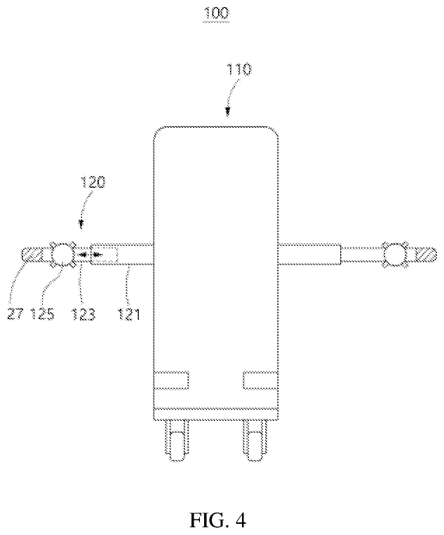

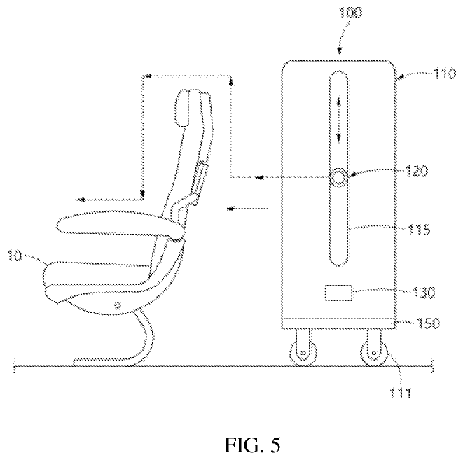

FIG. 1 is a diagram schematically illustrating the case where a robot for automatically preventing epidemics according to an exemplary embodiment of the present invention is used in a cabin of an airplane, FIG. 2 is a diagram illustrating a schematic configuration of the robot for automatically preventing epidemics illustrated in FIG. 1, FIG. 3 is a diagram illustrating a tilting operation of an injection unit of the robot for automatically preventing epidemics illustrated in FIG. 2, FIG. 4 is a diagram illustrating a sliding movement operation of the injection unit of the robot for automatically preventing epidemics illustrated in FIG. 2, and FIG. 5 is a diagram illustrating a side surface of the robot for automatically preventing epidemics illustrated in FIG. 2, and schematically illustrates a principle of a movement between seats.

As illustrated in the drawings, the robot 100 for automatically preventing epidemics according to the exemplary embodiment of the present invention is, for example, a robot for automatically performing epidemic prevention on an interior space IS of an airplane 1, and may include a moving body 110 movable along an in-flight passage of the airplane 1, injection units 120 which is mounted to both sides of the moving body 110 and injects a chemical solution for disinfection toward a seat 10 disposed in the cabin, an additional injection unit 130 which is mounted to the moving body 110 and injects a chemical solution toward a lower space of the seat 10, and an ultraviolet ray irradiation unit 150 which is mounted to a lower end of the moving body 10 and irradiates ultraviolet rays toward a floor of the passage.

Further, the robot 100 for automatically preventing epidemics may include a detection unit 127 which detects an external environment for appropriate operations of the moving body 110, the injection unit 120, and the like, and a control unit (not illustrated) which controls the operation of the moving body 110, the injection unit 120, and the like based on the degree of detection of the detection unit 127. The control unit may, for example, be linked with an application of a smart device of a user, and thus the user is capable of easily operating the robot 100 for automatically preventing epidemics of the present exemplary embodiment.

By the configuration, it is possible to efficiently and accurately prevent epidemics of the cabin. That is, the robot 100 for automatically preventing epidemics automatically performs epidemic prevention and disinfection work in the cabin, thereby reducing manpower consumption and securing efficiency and accuracy of the epidemic prevention.

Each configuration will be described. First, as illustrated in FIG. 1, the moving body 110 of the present exemplary embodiment has a width and a height so as to be allowed to pass between the passages in the cabin and enter between the seat 10 and the seat 10, and is mounted with a plurality of moving wheels 111 at a lower end for a smooth movement in a desired direction.

The moving body 110 may be provided in a cylinder shape or a rectangular prism shape, and is mounted with the moving wheels 111 at four parts of the lower end to be able to selectively move in any direction among front, rear, left, and right directions. The detection unit 127 which detects a distance from each part of an object, for example, the seat 10, disposed in the passage of the cabin may be mounted to the injection unit 120, which will be described below.

The control unit may control the movement of the moving body 110 based on detection information obtained by the detection unit 127, and for example, the control unit may control a rotation speed, a movement direction, and the like of the movement wheel 111.

The detection unit 127 may be provided in an infrared ray sensor. However, the type of detection unit 127 is not limited thereto.

In the meantime, the injection units 120 of the present exemplary embodiment may be provided in one pair and mounted to both sides of the moving body 110 as illustrated in FIGS. 1 and 2. Each injection unit 120 may be individually operated. The injection unit 120 may disinfect the seat 10 as a whole by injecting the chemical solution to the backrest part and the like including the part of the seat 10 on which the customer sits.

Referring to FIG. 2, the injection unit 120 of the present exemplary embodiment may include a first arm 121 mounted to a lateral wall of the moving body 110 and a second arm 123 mounted to be slidably movable with respect to the first arm 121 so as to be able to slide in a horizontal direction above the seat 10. Further, the detection unit 127 which detects a distance from the external object, for example, the seat 10, may be mounted to an end of the second arm 123 as described above.

Referring to FIGS. 1 and 2, an injection nozzle 125 for injecting the chemical solution for disinfection may be mounted to the second arm 123. The injection nozzle 125 may be provided with a plurality of injection holes through which the chemical solution is injected, and thus the epidemic prevention of the seat 10 may be reliably performed by injecting the chemical solution to the backrest and seat parts, and the like of the seat 10.

Referring to FIG. 3, the injection unit 120 is capable of tilting upward and downward with respect to the connection part with the moving body 110 as an axis. In other words, the first arm 12s in the injection unit 120 is mounted to be tiltable with respect to the moving body 110, and thus the control unit may adjust a tilting angle of the injection unit 120 with respect to the moving body 110 based on the detection information of the detection unit 127.

For example, the robot 100 for automatically preventing epidemics of the present exemplary embodiment moves forward, and in this case, the detection unit 127 mounted to the injection unit 120 may detect a gap with the seat 10 and the like in real time, and when a tilting operation of the injection unit 120 is required, the control unit may adjust a position of the injection unit 120 in real time by, for example, rotating a rotary motor which rotates the injection unit 120.

That is, by the tilting operation of the injection unit 120, it is possible to prevent the injection unit 120 from colliding with an external object, and the injection unit 120 may be located at the best position to provide the chemical solution to the seat 10.

In the meantime, referring to FIGS. 2 and 4, the second arm 123 of the injection unit 120 is slidably movable with respect to the first arm 121. As described above, based on the information of the detection unit 127 mounted to the end of the second arm 123, the control unit may adjust the position of the second arm 123 with respect to the first arm 121.

Further, referring to FIG. 5, the entire injection unit 120 may be slidably movable upward or downward with respect to the moving body 110. A guide member 115 for slidable movement of the injection unit 120 may be provided in the moving body 110 in a height direction.

The injection unit 120 has the structure of moving up and down along the guide member 115, so that when the robot 100 for automatically preventing epidemics of the present exemplary embodiment moves, the injection unit 120 may smoothly move while performing the epidemic prevention work without colliding with the seat 10 and the like.

As illustrated in FIG. 5, it is possible to detect a distance between the injection unit 120 and the seat 10 and the like in real time by the detection unit 127 mounted to the injection unit 120, and thus the control unit adjusts the position of the injection unit 120 and the injection unit 120 may move in a predetermined direction while injecting the chemical solution by using the injection nozzle 125.

As described above, the injection unit 120 is tiltable, the second arm 123 is slidably movable with respect to the first arm 121, and the whole injection unit 120 is movable in a vertical direction by the guide member 115 provided in the moving body 110, so that it is possible to prevent the injection unit 120 from colliding with the seat 10 and the like when the robot 100 for automatically preventing epidemics moves, and the injection unit 120 is capable of maintaining the best position in real time for a target for epidemic prevention.

In the meantime, the robot 100 for automatically preventing epidemics of the present exemplary embodiment may additionally include the additional injection unit 130 in order to perform the epidemic prevention on a lower surface of the seat 10, as illustrated in FIGS. 1 and 2.

The additional injection unit 130 of the present exemplary embodiment injects the chemical solution in a lateral direction when the moving body 110 moves, thereby disinfecting the lower space of the seat 10 in which a leg portion of a passenger is located when the passenger sits on the seat. The chemical solution is injected from the additional injection unit 120 in a radial direction, and thus it is possible to efficiently and accurately perform the epidemic prevention work on the lower space of the seat 10.

In the meantime, the robot 100 for automatically preventing epidemics of the present exemplary embodiment may further include the ultraviolet ray irradiation unit 150 which irradiates UV toward a floor part of the passage in the lower portion of the moving body 110 as illustrated in FIGS. 1 and 2.

The ultraviolet ray irradiation unit 150 may perform disinfection work on the floor portion by irradiating UV to the floor portion of the passage. However, the present exemplary embodiment has been described based on the case where the ultraviolet ray irradiation unit 150 is mounted toward the floor portion of the passage, but the ultraviolet ray irradiation unit 150 may also be mounted to another position of the moving body 110, and thus it is a matter of course that it is possible to perform the disinfection work on another portion.

As described above, according to the exemplary embodiment of the present invention, for example, it is possible to automatically perform the epidemic prevention work for the in-flight configuration, such as the seat 10, while the robot 100 for automatically preventing epidemics moves along the passage of the cabin, thereby achieving the advantage in improving efficiency and accuracy of the epidemic prevention work.

In the meantime, hereinafter, a robot for automatically preventing epidemics according to another exemplary embodiment of the present invention will be described with reference to the drawings, and the descriptions of the parts substantially the same as those of the robot for automatically preventing epidemics of the foregoing exemplary embodiment will be omitted.

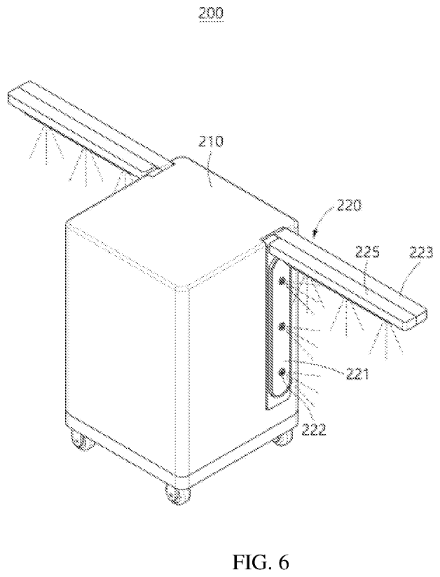

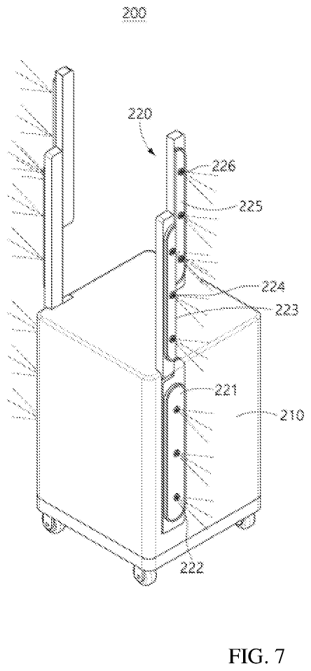

FIG. 6 is a perspective view schematically illustrating a configuration of a robot for automatically preventing epidemics according to another exemplary embodiment of the present invention, and FIG. 7 is a perspective view illustrating an operation of an injection unit illustrated in FIG. 6.

As illustrated in the drawings, a robot 200 for automatically preventing epidemics according to another exemplary embodiment of the present invention is different from the foregoing exemplary embodiment in the configuration of an injection unit 220.

The injection unit 220 of the exemplary embodiment may include first arms 221 mounted to both sides of a moving body 210, a second arm 223 tiltably coupled to an upper end of the first arm 221, and a third arm 225 coupled to the second arm 223 so as to be slidable with respect to the second arm 223, as illustrated in FIGS. 6 and 7.

Further, the plurality of injection nozzles 222, 224, and 226 is provided in the arms 221, 223, and 225 in a longitudinal direction, respectively, thereby injecting a chemical solution as a mist type in an injection direction.

The injection nozzles 222, 224, and 226 of the present exemplary embodiment are the nozzles for injecting the chemical solution as a mist in a fog type, and are different from the multi-directional injection nozzle 125 (see FIG. 3) of the foregoing exemplary embodiment. However, the present invention is not limited thereto.

Referring to FIG. 6, the first arms 221 may be mounted to both lateral walls of the moving body 210 and inject the chemical solution in both directions (horizontal direction) from the injection nozzles 222 mounted to the first arms 221, and in this case, an injection area by the injection nozzles 222 may be, for example, from a lower end of the seat to a center or an upper end of the seat, thereby smoothly performing the epidemic prevention work.

The second arm 223 has a structure capable of tilting with respect to the first arm 221, and thus as illustrated in FIG. 6, the second arm 223 may have an angle of 90 degrees with respect to the first arm 221, and as illustrated in FIG. 7, the second arm 223 may have an angle of 180 degrees with respect to the first arm 221. However, the second arm 223 may have another angle with respect to the first arm 221 as a matter of course.

Referring to FIG. 6, when the second arm 223 has the angle of 90 degrees with respect to the first arm 221, the injection direction of the injection nozzle 224 mounted to the second arm 223 is directed downward, and thus the chemical solution injected from the injection nozzle 224 is directed toward the seating part of the seat, thereby more accurately performing the epidemic prevention on the seat.

That is, the mist chemical solution is injected from the injection nozzle 222 of the first arm 221 in the horizontal direction and the mist chemical solution is injected from the injection nozzle 224 of the second arm 223 in the vertical direction, so that it is possible to more efficiently and accurately perform the epidemic prevention on the space of the cabin.

In the meantime, the robot 200 for automatically preventing epidemics of the present exemplary embodiment moves, the second arm 223 may interfere with the seat, and in order to prevent interference, the second arm 223 may be tilted so as to have the angle of 180 degrees with respect to the first arm 221. That is, by the detection information of the detection unit, the location of the second arm 223 with respect to the first arm 221 may be appropriately adjusted.

Further, as illustrated in FIG. 7, the third arm 225 has the structure that is capable of sliding (linearly moving) with respect to the second arm 223, thereby more expanding the range of the epidemic prevention. Further, based on the detection information of the detection unit, it is possible to variously adjust the locations of the second arm 223 and the third arm 225, so that it is possible to prevent the robot 200 for automatically preventing epidemics from interfering with the configurations of the cabin when the robot 200 for automatically preventing epidemics moves.

In the foregoing exemplary embodiments, the case where the robot for automatically preventing epidemics is applied to the space of the cabin of the airplane has been described, but the present invention is not limited thereto, and is applicable to, for example, another transportation means, a train or a performance hall with seats, and the like as a matter of course.

Although the specific exemplary embodiment according to the present invention has been described in the above, various modifications are possible without departing from the scope of the present invention. Therefore, the scope of the present invention shall not be determined while being limited to the foregoing exemplary embodiment, and the scope of the present invention shall be determined by the matters equivalent to the claims, as well as the accompanying claims.

While this invention has been described in connection with the limited exemplary embodiments and drawings, the present invention is not limited to the exemplary embodiment, and various changes and modifications are possible based on the descriptions by those skilled in the art. Therefore, the spirit of the present invention shall be recognized only by the accompanying claims, and it will be considered that all of the equivalent matters or the equivalent modifications belong to the scope of the spirit of the present invention.

1-12. (Canceled)

13. A robot for automatically preventing epidemics, the robot comprising:
a moving body which is movable along a passage of an interior space having a seat;
an injection unit which is mounted to the moving body and injects a chemical solution for disinfection toward the seat disposed in the interior space; and
an ultraviolet ray irradiation unit which is mounted to a lower end of the moving body and irradiates ultraviolet rays toward a floor of the passage,
wherein the robot for automatically preventing epidemics automatically performs epidemic prevention work in the interior space.

14. The robot of claim 13, wherein the injection unit includes a first arm mounted to the moving body so as to be slidable in a horizontal direction above the seat, and a second arm slidably movable with respect to the first arm, and
the second arm is provided with an injection nozzle which injects the chemical solution toward the seat in a radial direction.

15. The robot of claim 13, wherein the injection unit is tiltable in an upper direction and a lower direction with respect to a connection part with the moving body as an axis.

16. The robot of claim 13, wherein the moving body is provided with a guide member which guides the movement of the injection unit so that the injection unit is movable in an upper direction or a lower direction with respect to the moving body.

17. The robot of claim 13, wherein the injection unit includes:
first arms mounted to both sides of the moving body; and
a second arm tiltably coupled to an upper end of the first arm, and
the first arm and the second arm are provided with a plurality of injection nozzles in a longitudinal direction.

18. The robot of claim 17, wherein the injection unit further includes a third arm coupled to the second arm so as to be slidable with respect to the second arm, and
the third arm is provided with a plurality of injection nozzles in a longitudinal direction.

19. The robot of claim 13, wherein the moving body is mounted with a plurality of moving wheels at a lower end.

20. The robot of claim 13, wherein the injection units are provided in two and symmetrically mounted to both lateral walls of the moving body, and each of the injection units is individually operable.

21. The robot of claim 13, further comprising:
a detection unit which is mounted to the injection unit and detects a distance from an object disposed in the passage; and
a control unit which controls an operation of the moving body or the injection unit based on detection information obtained by the detection unit.

22. The robot of claim 21, wherein the detection unit is mounted to the injection unit, and
the control unit controls a tilting operation of the injection unit with respect to the moving body, a sliding operation of another portion with respect to one portion of the injection unit, and a sliding operation in a height direction of the injection unit with respect to the moving body based on information detected by the detection unit.

23. The robot of claim 13, further comprising:
an additional injection unit which is mounted to the moving body and injects a chemical solution to a space between the seats provided in the passage.

24. The robot of claim 13, wherein the robot automatically performs epidemic prevention work in a cabin space of an airplane, a transportation means with seats, or a performance hall with seats.
